(19) United States
(12) Patent Application Publication
(10) Pub. No.: US 2021/0095336 A1
(43) Pub. Date: Apr. 1, 2021

(54) METHODOLOGY FOR REAL-TIME VISUALIZATION OF GENOMICS-BASED ANTIBIOTIC RESISTANCE PROFILES

(71) Applicant: KONINKLIJKE PHILIPS N.V., Eindhoven (NL)

(72) Inventors: Joshua Keala Loving, Avon, MA (US); Helen Cecile van Aggelen, Cambridge, MA (US)

(21) Appl. No.: 17/019,800

(22) Filed: Sep. 14, 2020

Related U.S. Application Data
(60) Provisional application No. 62/907,974, filed on Sep. 30, 2019.

(51) Int. Cl. C12Q 1/6869; G16B 40/00

(52) U.S. Cl. CPC C12Q 1/6869; G01N 2800/44; G16B 40/00

(57) ABSTRACT

A wherein the processor is further configured to method of determining the antibiotic resistance of a pathogen from in a sample from a patient or the environment in real-time, including: sequencing the genome of the pathogen from the patient sample in real-time; identifying the pathogen from the sequencing data as it becomes available; determining if the pathogen is resistant to a first antibiotic using a machine learning model, the identity of the pathogen, and the sequencing data for the pathogen; tabulating data regarding the antibiotic resistance of the pathogen; and producing a graphical user interface indicating the antibiotic resistance of the pathogen

### FIELD

This invention relates generally to identifying genes conferring antibiotic resistance in a pathogen infecting a patient, and recommending treatment protocols based on the presence or absence of antibiotic resistance.

### BACKGROUND

Traditionally, infections arising from bacterial pathogens are treated with antibiotics. However, various pathogenic bacterial strains, as well as strains of other pathogens, have evolved resistance to include genes conferring resistance to certain antibiotics in their genome.

When a patient presents with an infection, the resistance profile of the pathogen is traditionally determined by culturing the pathogen and exposing it to varying concentrations of antibiotics to determine the minimum inhibitory concentration (MIC). The process of culture and phenotypic assay of drug resistance (MIC test) is too slow to inform the initial treatment of the patient.

Genetic sequencing of the infectious pathogen can be done, and the genetic sequences can be analyzed for presence of antibiotic resistance genes. For example, Staphylococcus aureus infections can be sequenced, and examined for the presence of the mecA gene conferring resistance to beta-lactam antibiotics. Results from sequencing of the genome may be used to modify the initial treatment of the patient.

A method is needed to develop methods of rapidly identifying the presence or absence of antibiotic resistance in a pathogen.

### SUMMARY OF THE INVENTION

The current disclosure is directed to a method of recommending a treatment protocol for a patient infected by a pathogen. In various embodiments, the method of recommending a treatment protocol includes identifying a plurality of resistance genes; and preparing a first database associating each resistance gene with resistance to a first antibiotic and a second antibiotic.

Various embodiments relate to a method of determining the antibiotic resistance of a pathogen from in a sample from a patient or the environment in real-time, including: sequencing the genome of the pathogen from the patient sample in real-time; identifying the pathogen from the sequencing data as it becomes available; determining if the pathogen is resistant to a first antibiotic using a machine learning model, the identity of the pathogen, and the sequencing data for the pathogen; tabulating data regarding the antibiotic resistance of the pathogen; and producing a graphical user interface indicating the antibiotic resistance of the pathogen.

Various embodiments are described, wherein the machine learning model is a classification model indicating whether the pathogen is resistant to the first antibiotic.

Various embodiments are described, wherein the machine learning model is a regression model that produces a minimum inhibitory concentration (MIC) value for the pathogen that is then compared to a breakpoint value to determine if the pathogen is resistant to the first antibiotic.

Various embodiments are described, wherein the machine learning model determines if the pathogen is resistant to a plurality of antibiotics.

Various embodiments are described, further including training the machine learning model.

Various embodiments are described, wherein the machine learning model is modified using adaptive training techniques when new pathogen training data is available.

Various embodiments are described, further including producing a notification once the real-time sequencing reaches a check criteria.

Various embodiments are described, wherein check criteria is a confidence interval threshold.

Various embodiments are described, wherein check criteria includes both a time limit and a confidence interval threshold.

Various embodiments are described, wherein the notification is one of a visual indicator on the graphical user interface, an audible alert, an electronic message, a text message, and an email message.

Various embodiments are described, wherein graphical user interface includes an indication using a color scheme to indicate the pathogen's level of resistance to the first antibiotic.

Various embodiments are described, wherein the color is selected based upon a set of thresholds and the number of sequencing reads associated with resistance against the first antibiotic.

Various embodiments are described, wherein tabulating data regarding the antibiotic resistance of the pathogen includes tabulating data for a plurality of different pathogens and a plurality of different antibiotics.

Various embodiments are described, further including updating the graphical representation as more sequencing data becomes available.

Further various embodiments relate to a system for determining the antibiotic resistance of a pathogen from in a sample from a patient or the environment in real-time, including: a sequencing apparatus for generating a plurality of readings of the genome of the pathogen from the patient sample in real-time; and a processor configured to: identify the pathogen from the sequencing data as it becomes available; determine if the pathogen is resistant to a first antibiotic using a machine learning model, the identity of the pathogen, and the sequencing data for the pathogen; tabulate data regarding the antibiotic resistance of the pathogen; and producing a graphical user interface indicating the antibiotic resistance of the pathogen.

Various embodiments are described, wherein the machine learning model is a classification model indicating whether the pathogen is resistant to the first antibiotic.

Various embodiments are described, wherein the machine learning model is a regression model that produces a minimum inhibitory concentration (MIC) value for the pathogen that is then compared to a breakpoint value to determine if the pathogen is resistant to the first antibiotic.

Various embodiments are described, wherein the machine learning model determines if the pathogen is resistant to a plurality of antibiotics.

Various embodiments are described, wherein the processor is further configured to train the machine learning model.

Various embodiments are described, wherein the machine learning model is modified using adaptive training techniques when new pathogen training data is available.

Various embodiments are described, wherein the processor is further configured to produce a notification once the real-time sequencing reaches a check criteria.

Various embodiments are described, wherein check criteria is a confidence interval threshold.

Various embodiments are described, wherein check criteria includes both a time limit and a confidence interval threshold.

Various embodiments are described, wherein the notification is one of a visual indicator on the graphical user interface, an audible alert, an electronic message, a text message, and an email message.

Various embodiments are described, wherein graphical user interface includes an indication using a color scheme to indicate the pathogen's level of resistance to the first antibiotic.

Various embodiments are described, wherein the color is selected based upon a set of thresholds and the number of sequencing reads associated with genetic resistance against the first antibiotic.

Various embodiments are described, wherein tabulating data regarding the antibiotic resistance of the pathogen includes tabulating data for a plurality of different pathogens and a plurality of different antibiotics.

Various embodiments are described, wherein the processor is further configured to update the graphical representation as more sequencing data becomes available.

### BRIEF DESCRIPTION OF THE DRAWINGS

In order to better understand various exemplary embodiments, reference is made to the accompanying drawings, wherein:

FIG. 1 illustrates a flow diagram of a method 100 for determining in real-time the presence of antibiotic resistance of a pathogen in a patient sample;

FIGS. 2A, 2B, and 2C illustrate a plot of the total number of reads assigned to each type of pathogen detected in the patient sample;

FIGS. 3A, 3B, and 3C illustrate a graphical user interface showing the number of sequencing reads associated with antibiotic resistance via a model for each type of pathogen detected; and

FIG. 4 illustrates an exemplary hardware diagram for implementing the method.

### DETAILED DESCRIPTION

The extensive use of antibiotics in medicine and agriculture, coupled with a shortage of new antibiotics, has given rise to an antibiotic resistance crisis, in which several pathogens are becoming resistant to treatment. Hospital-acquired infections are a major concern for both hospitals and patients, especially when the infection involves a multi-drug resistant pathogen. Not only do hospital-acquired infections put patients at risk, they also put a big financial and reputational burden on the hospital and health care system. Additionally, different regions, e.g., different states or contries, may have very different incidences of drug resistance in the same species of pathogen. Thus, an antibiotic may be effective against a pathogen in one area, but ineffective against an infection by the same species of pathogen in a different area.

Various embodiments disclosed herein relate to a system for recommending a treatment protocol for a patient infected by a pathogen. In various embodiments, the system includes a first database storing a plurality of resistance genes, where each resistance gene is classified as conferring resistance to a particular antibiotic. As an example, the database may include resistance genes from Staphylococcus aureus, where a first resistance gene confers resistance to ampicillin on the pathogen, a second resistance gene confers resistance to azithromycin, and a third resistance gene confers resistance to clindamycin.

In various embodiments, the system may also include a sequencing apparatus for generating a plurality of readings of the genome of the pathogen infecting the patient. The sequencing apparatus may be configured to generate a plurality of readings in real time. A suitable sequencing apparatus may include an apparatus for Single Molecule, Real-Time (SMRT) Sequencing. Alternatively, the sequencing apparatus may provide protein nanopore sequencing.

SMRT sequencing is carried out on a chip that contains many zero-mode waveguides (ZMW) illuminated by a laser. Each ZMW includes an immobilized DNA polymerase molecule which binds to a molecule of single stranded DNA and free nucleotide bases. A corresponding fluorescent dye molecule is bound to each type of nucleotide base, e.g., adenosine may be labeled with a first fluorescent dye and cytosine may be labeled with a second fluorescent dye, where the first and second dyes have different colors. As each nucleotide is incorporated into a newly synthesized DNA molecule by the DNA polymerase, a detector records a characteristic fluorescent emission for the labeled base. The sequence of emissions may be used to determine the DNA sequence in real time, as the DNA is replicated.

In protein nanopore sequencing, an electric current is passed through a protein nanopore in an electrically resistant polymer membrane. As a DNA strand passes through the pore, each base creates a characteristic disruption in the recorded current. Measurement of changes in current allows identification of each base as it passes through the nanopore.

Various other sequencing techniques may be used as well, that are able to provide real-time information regarding the reading of sequences.

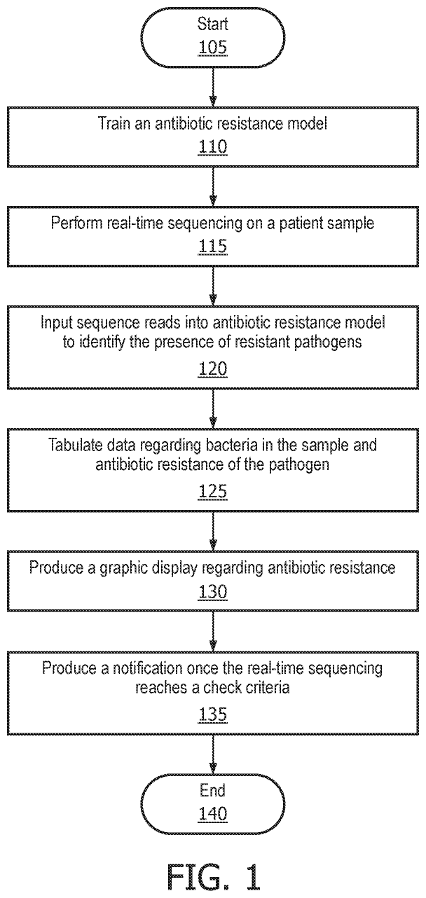

FIG. 1 illustrates a flow diagram of a method 100 for determining in real-time the presence of antibiotic resistant pathogens in a patient sample. The method 100 may be carried out by a processor. First, an antibiotic resistance model is developed and trained 110. This model receives as inputs the type of pathogen detected and the genetic makeup of the pathogen that may indicate the presence of resistance genes and/or other virulence factors and/or other sequences associated with antibiotic resistance. The model may be a classification model that outputs an indication of whether the detected pathogen is resistant to the various antibiotics. The model may be a regression model that produces an MIC value that indicates the concentration of antibiotic needed to prevent pathogen growth. This MIC value may be compared to breakpoints to determine if the detected pathogen is resistant to a specific antibiotic. The breakpoints may vary by site, facility, or practitioner. Further, the breakpoints may be based upon various standards, for example, the European Committee on Antimicrobial Susceptibility Testing (EUCAST) breakpoints. The output of the model may be called a resistance indicator.

The antibiotic resistance model may be trained using training data obtained by testing a wide variety of genetic variants of different pathogens of interest and their response to various concentrations of antibiotics. The model may be one of any of a variety of known machine learning models such as deep learning neural networks, support-vector machines (SVM), etc. As mentioned above, the model may be a classification model that produces a resistance indicator that is an indication of resistance for a selected set of antibiotics. Also, the model may be a regression model that produces an MIC value for each antibiotic that is then compared to break points that then indicate whether the detected pathogen is resistant to each antibiotic to produce the resistance indicator. Any type of regression model may be used.

The model may be trained to indicate antibiotic resistance for a specific antibiotic based upon specific resistance genes found in the pathogen. Such a model will detect the presence of one or more resistance genes and indicate the associated antibiotic resistance. In other models, the antibiotic resistance is based upon the combination of genomic sequences found in the detected pathogen. Such a model will detect antibiotic resistance that results from specific combinations of genomic variations.

The model may also be updated using adaptive learning techniques as new training data becomes available. This allows the model to evolve as new variants of pathogen are discovered and tested. Alternatively, as new data is received the model may be completely retrained using the new data.

Next, a patient sample is taken and real-time genetic sequencing is done on the sample 115. As described above, the real-time genetic sequencing may be done using various known techniques. The real-time sequencing data is processed to identify the presence of various types of pathogens and the specific genetic variations found in the detected pathogen.

As the sequencing data for the various detected pathogens are produced, it is input into the antibiotic resistance model to determine if the detected pathogen is resistant to various antibiotics 120. The outputs of the antibiotic resistance model are tabulated 125 and then used to produce a graphic display regarding antibiotic resistance.

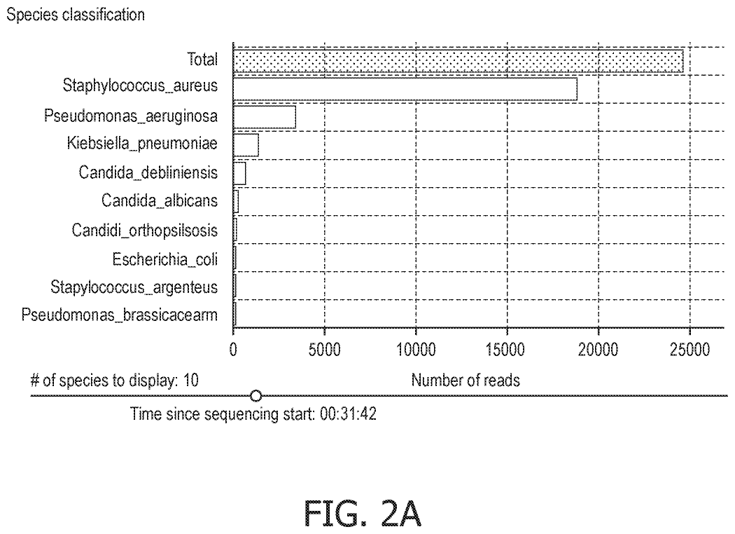

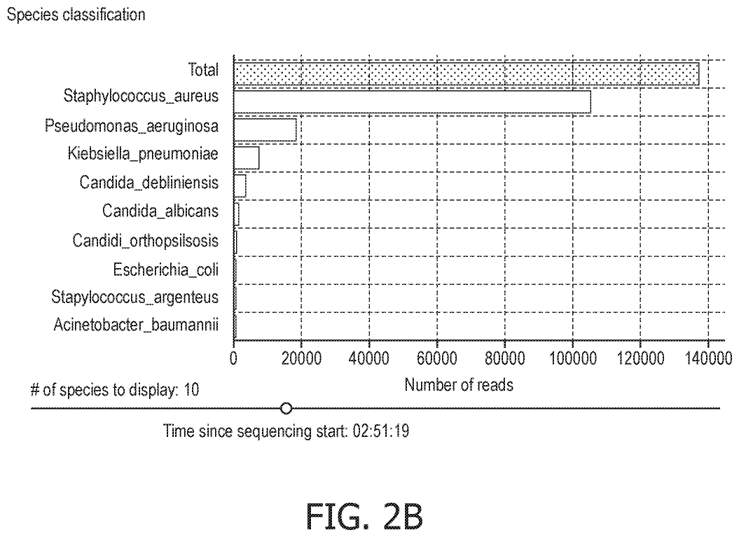

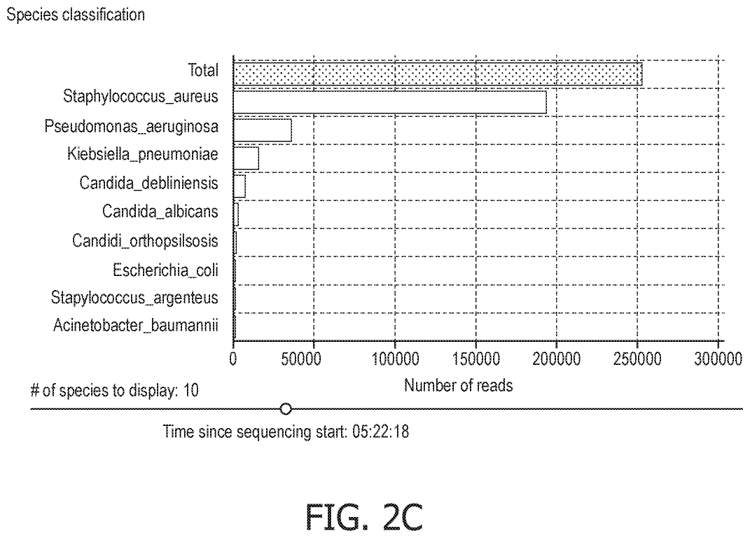

FIGS. 2A, 2B, and 2C illustrate a plot the number of reads for each type of pathogen detected in the patient sample. Each figure shows the number of reads at a different elapsed sequencing time. The top bar shows the total number of reads. Then below are individual bars showing the number of reads indicating various types of pathogens found in the sample. As expected, the number of reads increases as time passes. Such a graphical display provides a caregiver an indication of the types of pathogens found in the patient.

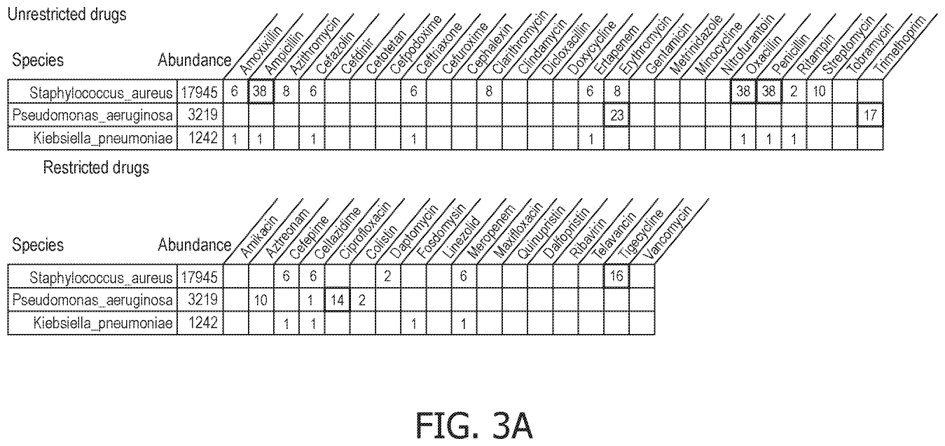

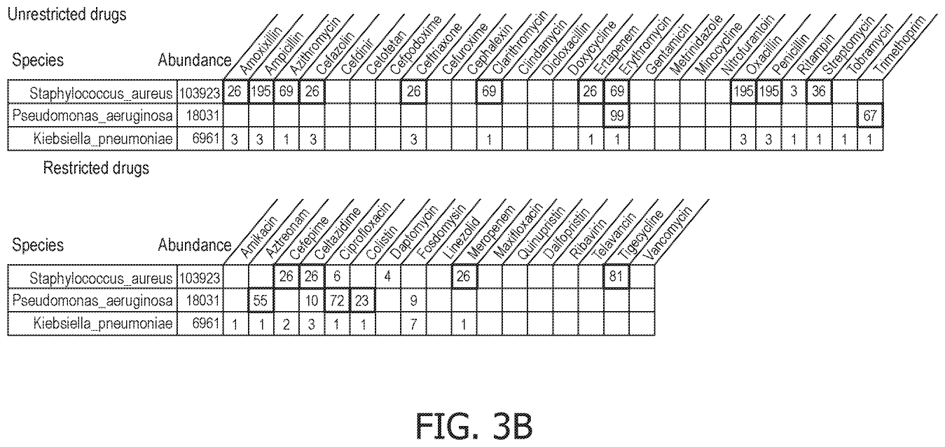

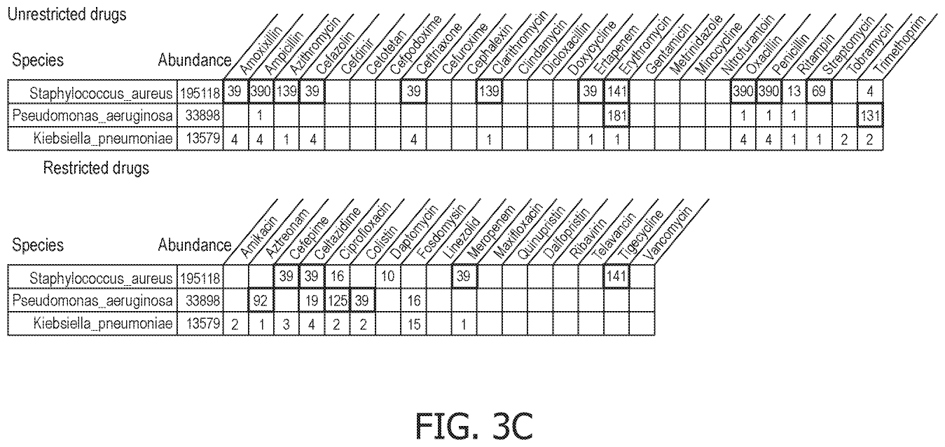

FIGS. 3A, 3B, and 3C illustrate graphical user interface showing the presence of various antibiotic resistant variations of each type of pathogen detected. In this example, three different types of pathogen have been detected: Staphylococcus aureus; Pseudomonas aeruginosa; and Klebsiella pneumoniae. The graphical representation is shown as a table with a row for each pathogen detected and a column for each antibiotic drug of interest. In this example, two tables are shown that split the antibiotics into two different groups: unrestricted drugs and restricted drugs. In other embodiments such a separation may not be used. A second column in the table indicates the abundance of each detected pathogen. Specifically, it is the total number of reads that indicate the presence of each pathogen. Further, each element of the table then shows the number of reads for the pathogen (row) that indicate a resistance to the antibiotic (column). Statistical analysis may then be done for each table entry to compare the value to a threshold value and to determine a confidence interval for the value. This statistical analysis may then be used to add a visual shading where one direction on a color scale indicates antibiotic sensitivity and the other direction indicates antibiotic resistance. This representation and use of colors will allow a caregiver to quickly assess which antibiotics will be best suited to treat the patient's infection. In some situations, such that that shown in FIGS. 3A, 3B, and 3C, there may be a plurality of antibiotics that indicate sensitivity for each of the detected pathogens. In other situations, no one antibiotic will be effective against all of the pathogens present in a patient, but two or more antibiotics may be used to effectively treat each of the pathogens present. In another embodiment of the graphical user interface, the number of reads showing genetic resistance to the antibiotic may not be shown, and only the color shading is used to communicate to the caregiver whether a specific antibiotic will be effective against a specific pathogen. While tables are shown to present the data, other visual elements may be used as well to present the antibiotic resistance information to the caregiver.

As the real-time sequencing occurs, the graphical user interface is updated with the most recent data. The question is then: when have enough reads been processed to make a treatment decision? This may be done by setting a check criteria. Such a criteria may be a confidence threshold. As the data is collected, statistical analysis of the data may be performed to determine the confidence interval of the various data collected. This may then be compared to a confidence threshold. Once the confidence threshold is exceeded, a notification may be sent to the caregiver via the graphical user interface or may be communicated to the caregiver using an electronic method such as via text message, email, using an audible alarm, or any other such method. Another check criteria may be a simple timer. That is set a specific time and then alert the caregiver to view the graphical user interface and make a treatment decision. In another embodiment, both the confidence threshold and a timer may be used. When either of the criteria are met, then the notification for the caregiver is generated. The values for the check criteria may be automatically selected based upon various factors such as the severity of the infection, the specific facility, or knowledge of recent infection outbreaks. Also, the values for the check criteria may be determined by the caregiver.

For example, there is a tradeoff between the severity of the infection and the amount of time it will take to acquire sufficient reads to make an informed treatment decision. When the infection is more severe, and the caregiver is concerned that the patient may deteriorate quickly, the check criteria are loosened so that they are met in shorter amount of time. For example, a normal confidence threshold may be 80%, but for a patient with a severe infection, it is desired to wait no longer than hour to make a treatment decision. In such a case, the confidence threshold may be set to 80% along with a 1 hour time limit. As a result, if the confidence threshold is met in less than an hour, then the treatment decision may be made sooner. If at the one hour time, the confidence level has not been reached, then the caregiver can at that time decide to make a treatment decision based upon the available data or to wait a bit longer. Alternatively, the confidence threshold may be set to 60%. This allows for the caregiver to tailor the check criteria based upon the patient's current condition.

After the initial course of antibiotics is administered to the patient, the caregiver may continue to periodically monitor the graphical user interface to determine that no other genetic variants of pathogen have been found that would be resistant to the current treatment or that no new pathogens have been detected. If so, then the caregiver may modify the treatment as needed to treat the pathogen found in the sample. In order to assist the caregiver, the graphical user interface may highlight new pathogens that were detected since the notification was sent or any other significant changes in the data presented.

As the patient receives treatment, it is possible to take a second sample from the patient and start the process over again to determine the current pathogen that are causing the patient's infection. This allows the caregiver to see that treatment is indeed decreasing the presence of the infection and allows for further detection of pathogen and/or pathogen resistances that the patient may newly have acquired. In such a case, the data from the first sample may be presented along with data from the second sample. The graphical user interface may then highlight differences between the two samples so make it easier for the caregiver to note the changes.

In some situations, when the patient presents with a severe infection, the caregiver may start the patient on a course of antibiotics based upon their judgement and experience and any information available to the caregiver that would indicate a certain type of pathogen. The present treatment course may then be verified or modified based upon the results from the processing of the real-time sequencing data results.

Using current sequencing techniques, sufficient sequence reads may be obtained in 10 to 30 minutes or 30 to 60 minutes that would provide sufficient confidence that a caregiver may provide a recommendation. In other situations, it may take longer. In any case, the complete sequencing would typically be completed in 30 minutes to 10 hours based upon currently available technologies.

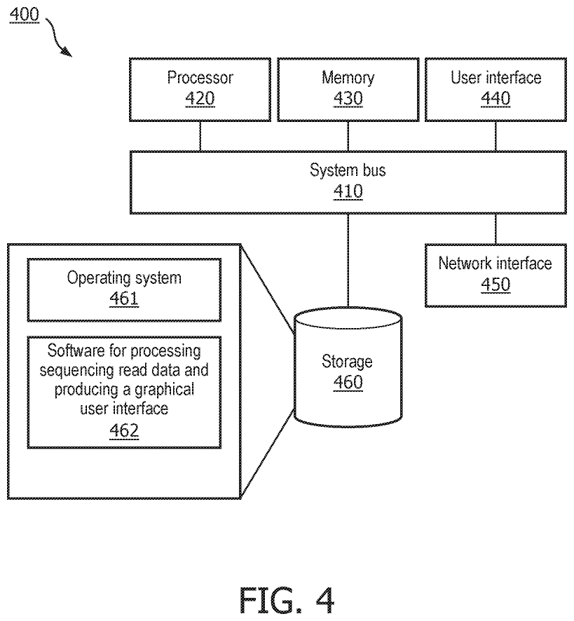

FIG. 4 illustrates an exemplary hardware diagram 400 for implementing the method 100. As shown, the device 400 includes a processor 420, memory 430, user interface 440, network interface 450, and storage 460 interconnected via one or more system buses 410. It will be understood that FIG. 4 constitutes, in some respects, an abstraction and that the actual organization of the components of the device 400 may be more complex than illustrated.

The processor 420 may be any hardware device capable of executing instructions stored in memory 430 or storage 460 or otherwise processing data. As such, the processor may include a microprocessor, microcontroller, graphics processing unit (GPU), field programmable gate array (FPGA), application-specific integrated circuit (ASIC), or other similar devices.

The memory 430 may include various memories such as, for example L1, L2, or L3 cache or system memory. As such, the memory 430 may include static random-access memory (SRAM), dynamic RAM (DRAM), flash memory, read only memory (ROM), or other similar memory devices.

The user interface 440 may include one or more devices for enabling communication with a user such as an administrator. For example, the user interface 440 may include a display, a touch interface, a mouse, and/or a keyboard for receiving user commands. In some embodiments, the user interface 440 may include a command line interface or graphical user interface that may be presented to a remote terminal via the network interface 450. The user interface 440 may implement the graphical user interface presented to the user of the system described above in FIGS. 1, 2A, 2B, 2C, 3A, 3B, and 3C.

The network interface 450 may include one or more devices for enabling communication with other hardware devices. For example, the network interface 450 may include a network interface card (NIC) configured to communicate according to the Ethernet protocol or other communications protocols, including wireless protocols. Additionally, the network interface 450 may implement a TCP/IP stack for communication according to the TCP/IP protocols. Various alternative or additional hardware or configurations for the network interface 450 will be apparent.

The storage 460 may include one or more machine-readable storage media such as read-only memory (ROM), random-access memory (RAM), magnetic disk storage media, optical storage media, flash-memory devices, or similar storage media. In various embodiments, the storage 460 may store instructions for execution by the processor 420 or data upon with the processor 420 may operate. For example, the storage 460 may store a base operating system 461 for controlling various basic operations of the hardware 400. The storage 461 may also include instructions for carrying out the method of processing sequencing read data and presenting antibiotic resistance information using the graphical user interface as described above.

It will be apparent that various information described as stored in the storage 460 may be additionally or alternatively stored in the memory 430. In this respect, the memory 430 may also be considered to constitute a “storage device” and the storage 460 may be considered a “memory.” Various other arrangements will be apparent. Further, the memory 430 and storage 460 may both be considered to be “non-transitory machine-readable media.” As used herein, the term “non-transitory” will be understood to exclude transitory signals but to include all forms of storage, including both volatile and non-volatile memories.

While the host device 400 is shown as including one of each described component, the various components may be duplicated in various embodiments. For example, the processor 420 may include multiple microprocessors that are configured to independently execute the methods described herein or are configured to perform steps or subroutines of the methods described herein such that the multiple processors cooperate to achieve the functionality described herein. Further, where the device 400 is implemented in a cloud computing system, the various hardware components may belong to separate physical systems. For example, the processor 420 may include a first processor in a first server and a second processor in a second server.

Currently testing a patient sample for antibiotic resistance takes many hours or days. When the patient has a severe infection, it is possible that a few hours of delay in treatment may lead to significant harm to the patient or even death. The embodiments of a method and system for real-time sequencing of a patient sample and processing the sequencing reads solves this technological problem by greatly reducing the time to determine the antibiotic resistance of the pathogen causing the patient illness. This is accomplished by processing the real-time sequencing read data to identify pathogen with genetic variations that lead to antibiotic resistance. This is done using a machine learning model that receives the sequencing read data in real-time and determines if the data indicates that the pathogen is antibiotic resistant. As the data is received and processed in real-time, this information may be presented to a caregiver who can make a treatment decision based upon the data. This can significantly reduce the time to providing a targeted antibiotic treatment based upon the patient sample.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method of determining the antibiotic resistance of a pathogen from in a sample from a patient or the environment in real-time, comprising:
sequencing the genome of the pathogen from the patient sample in real-time;
identifying the pathogen from the sequencing data as it becomes available;
determining if the pathogen is resistant to a first antibiotic using a machine learning model, the identity of the pathogen, and the sequencing data for the pathogen;
tabulating data regarding the antibiotic resistance of the pathogen; and
producing a graphical user interface indicating the antibiotic resistance of the pathogen.

2. The method of claim 1, wherein the machine learning model is a classification model indicating whether the pathogen is resistant to the first antibiotic.

3. The method of claim 1, wherein the machine learning model is a regression model that produces a minimum inhibitory concentration (MIC) value for the pathogen that is then compared to a breakpoint value to determine if the pathogen is resistant to the first antibiotic.

4. The method of claim 1, wherein the machine learning model determines if the pathogen is resistant to a plurality of antibiotics.

5. The method of claim 1, further comprising training the machine learning model.

6. The method of claim 5, wherein the machine learning model is modified using adaptive training techniques when new pathogen training data is available.

7. The method of claim 1, further comprising producing a notification once the real-time sequencing reaches a check criteria.

8. The method of claim 7, wherein check criteria is a confidence interval threshold.

9. The method of claim 7, wherein check criteria includes both a time limit and a confidence interval threshold.

10. The method of claim 7, wherein the notification is one of a visual indicator on the graphical user interface, an audible alert, an electronic message, a text message, and an email message.

11. The method of claim 1, wherein graphical user interface includes an indication using a color scheme to indicate the pathogen's level of resistance to the first antibiotic.

12. The method of claim 11, wherein the color is selected based upon a set of thresholds and the number of sequencing reads associated with resistance against the first antibiotic.

13. The method of claim 1, wherein tabulating data regarding the antibiotic resistance of the pathogen includes tabulating data for a plurality of different pathogens and a plurality of different antibiotics.

14. The method of claim 1, further comprising updating the graphical representation as more sequencing data becomes available.

15. A system for determining the antibiotic resistance of a pathogen from in a sample from a patient or the environment in real-time, comprising:
a sequencing apparatus for generating a plurality of readings of the genome of the pathogen from the patient sample in real-time; and
a processor configured to:
identify the pathogen from the sequencing data as it becomes available;
determine if the pathogen is resistant to a first antibiotic using a machine learning model, the identity of the pathogen, and the sequencing data for the pathogen;
tabulate data regarding the antibiotic resistance of the pathogen; and
producing a graphical user interface indicating the antibiotic resistance of the pathogen.

16. The system of claim 15, wherein the machine learning model is a classification model indicating whether the pathogen is resistant to the first antibiotic.

17. The system of claim 15, wherein the machine learning model is a regression model that produces a minimum inhibitory concentration (MIC) value for the pathogen that is then compared to a breakpoint value to determine if the pathogen is resistant to the first antibiotic.

18. The system of claim 15, wherein the machine learning model determines if the pathogen is resistant to a plurality of antibiotics.

19. The system of claim 15, wherein the processor is further configured to train the machine learning model.

20. The system of claim 19, wherein the machine learning model is modified using adaptive training techniques when new pathogen training data is available.

21. The system of claim 15, wherein the processor is further configured to produce a notification once the real-time sequencing reaches a check criteria.

22. The system of claim 21, wherein check criteria is a confidence interval threshold.

23. The system of claim 21, wherein check criteria includes both a time limit and a confidence interval threshold.

24. The system of claim 21, wherein the notification is one of a visual indicator on the graphical user interface, an audible alert, an electronic message, a text message, and an email message.

25. The system of claim 15, wherein graphical user interface includes an indication using a color scheme to indicate the pathogen's level of resistance to the first antibiotic.

26. The system of claim 15, wherein the color is selected based upon a set of thresholds and the number of sequencing reads associated with genetic resistance against the first antibiotic.

27. The system of claim 15, wherein tabulating data regarding the antibiotic resistance of the pathogen includes tabulating data for a plurality of different pathogens and a plurality of different antibiotics.

28. The system of claim 15, wherein the processor is further configured to update the graphical representation as more sequencing data becomes available.
